(19) United States
(12) Patent Application Publication
(10) Pub. No.: US 2015/0031932 A1
(43) Pub. Date: Jan. 29, 2015

(54) Method to Accurately and Rapidly Locate Tumor

(71) Applicant: Nanjing Chengda MedicalTechnology Co., Ltd., Nanjing (CN)

(72) Inventors: Yun Ge, Nanjing (CN); Jie Zhang, Nanjing (CN); Yue Guan, Nanjing (CN)

(21) Appl. No.: 14/340,484

(22) Filed: Jul. 24, 2014

(30) Foreign Application Priority Data
Jul. 24, 2013 (CN) 201310312649.2

(51) Int. Cl. A61N 5/10; A61B 6/03

(52) U.S. Cl. CPC A61N 5/1049; A61B 6/032; A61N 2005/1051; A61N 2005/1097
USPC 600/1

(57) ABSTRACT

A method to accurately and rapidly locate tumor, comprising the steps of using a immobilization module to ensure the consistency of patient positioning, verification and the position of each treatment; using a locating and marking module comprising a locating and marking tool for locating and marking; using a data input module to obtain the data required for a calculation of Optical Positioning System in radiotherapy; and using a positioning module to calculate the deviation between the planned isocenter and the machine isocenter, and to provide prompts of patient positioning.

### FIELD OF THE INVENTION

The present invention pertains to a method to accurately and rapidly locate tumors accurately, and in particular relates to a method that can track the position of the planned isocenter in real time and accurately and quickly calculate the deviation between the planned isocenter and the machine isocenter.

### BACKGROUND OF THE INVENTION

Radiotherapy is an important method in cancer treatment. It has been widely adopted for its simplicity, significant effect on prolonging patient's life.

In precision radiotherapy, there is a need to accurately locate the tumor. In practice, the inaccuracies caused by radiotherapy equipment, such as room lasers, simulator, accelerator, and manual operation, make it difficult to guarantee the quality of precision radiotherapy and affect the effectiveness of the treatment of cancer patients directly. To reduce these inaccuracies, it is necessary to track the position of the planned isocenter in real time and to calculate the deviation between the planned isocenter and the machine isocenter during the entire course of patient positioning to guide radiotherapist to locate target properly.

To solve the problems described above, an accurate and quick method that can track the real-time position of the planned isocenter and calculate the deviation between the planned isocenter and the machine isocenter has become essential.

### SUMMARY OF THE INVENTION

To correct the deficiencies in the present technologies, the present invention provides a method to accurately and rapidly locate tumors . It can track the real-time position of the planned isocenter accurately and quickly. In addition, the deviation between the planned isocenter and the machine isocenter can be calculated in real time.

To solve the technical problems above, the technical solution of the invention is implemented as follows:

The method to accurately and rapidly locate tumors disclosed in the present invention can comprise steps of using an immobilization module, which can be used to ensure the consistency of patient positioning, verification and the position of each treatment; using a locating and marking module, wherein the locating and marking tools can be mounted on auxiliary fixing devices for radiotherapy; using a data input module, which can be used to obtain the data required for the calculation of Optical Positioning System in radiotherapy; and using a positioning module, which can be used to calculate the deviation between the planned isocenter and the machine isocenter, and to provide prompts of patient positioning.

The invention discloses a method to accurately and rapidly locate tumors, which is significantly different from the conventional method.

The rapid method to locate tumors accurately comprises the following modules:
B1: Immobilization module;
B2: Locating and marking module;
B3: Data input module;
B4: Positioning module.

In one embodiment of the present invention, module B1 can comprise one or more of the following steps:
S1: Immobilization with mask:
Patients with brain or head and/or neck tumor are immobilized with masks and patients with body tumor are immobilized with body masks.
S2: Patient is immobilized with vacuum bags, body position fixing devices and other auxiliary fixing devices.

In one embodiment of the present invention, module B2 can comprise one or more of the following steps:
S3: One or more locating and marking tools are attached to the fixing devices and/or mask and/or body mask according to the condition of patients.

The locating and marking tools are attached to at least one of the following locations: 1) auxiliary fixing devices; 2) body mask; 3) face mask.

In at least one embodiment of the invention, the locating and marking tool mentioned is a positioning ball, however, ordinary person skilled in the art should know that the marking objects in other shapes or other materials with an obvious infrared signature can be substituted for the positioning ball. Therefore, similar technical solutions do not lie outside the range of the invention disclosure and claims.

The number of the locating and marking tools is preferably 6 or more to ensure that the translational or rotational changes of the locating and marking tools which when detected can fully indicate the changes of 6 degrees of freedom in three-dimensional space. If less than 6 locating and marking tools are used, the accuracy of the positioning can be insufficient. However, if more than 6 tools are used, the computational complexity will increase significantly although a higher accuracy can be obtained, making it impractical.

In one embodiment of the present invention, module B3 can comprise one or more of the following steps:
S4: Computed tomography images of patients with auxiliary fixing devices and/or mask and/or body mask and locating and marking tools are obtained by the medical imaging systems; the positions of the locating and marking tools, human body and auxiliary fixing devices are obtained by a medical imaging system.
S5: The computed tomography images obtained by S4 are imported into Treatment Planning System (TPS) to identify the planned isocenter and to produce a treatment plan;
S6: The treatment plan obtained by S5 is imported into an Optical Positioning System;
S7: The 3-D coordinates of the machine isocenter are obtained by a pen register, and are imported into the Optical Positioning System.

In one embodiment of the invention, module B4 can comprise one or more of the following steps:
S8: The Optical Positioning System uses the data of the treatment plan, the planned isocenter and the 3-D coordinates of the machine isocenter to calculate the deviation between the planned isocenter and the machine isocenter.
The Optical Positioning System mentioned in the invention can use the data of the treatment plan, the planned isocenter and the 3-D coordinates of the machine isocenter to calculate the deviation between the planned isocenter and the machine isocenter in real time or non-real time.
S9: The deviation between the planned isocenter and the machine isocenter is displayed on the Optical Positioning System, and the appropriate prompts of patient positioning are provided.

The prompts of patient positioning mentioned in the invention can comprise the manual operating instructions to instruct users how to manually adjust the parameters of the treatment table, the gantry, etc; or/and the automatic operating instructions to give an automatic positioning control device instructions to automatically adjust the parameters of the treatment couch, the gantry, etc.

### BENEFICIAL EFFECTS

The invention has the following advantages: as shown in FIG. 2 and FIG. 3, when compared to the conventional method, the method to accurately and rapidly locate tumors can simplify the procedure of the conventional method of patient positioning, and can greatly reduce the positioning errors caused by manual operations and repeated patient positioning. Consequently, it greatly improves the efficiency of tumor locating and lowers the work load of physicians and radiotherapists. Furthermore, the invention can provide real-time and non-real-time positioning correction instructions to users or an automatic positioning device to locate target properly, which improves the positioning accuracy of the entire course of patient positioning.

### BRIEF DESCRIPTION OF THE DRAWINGS

FIG. 1 is a flow chart illustrating the procedure of the method to accurately and rapidly locate tumor.

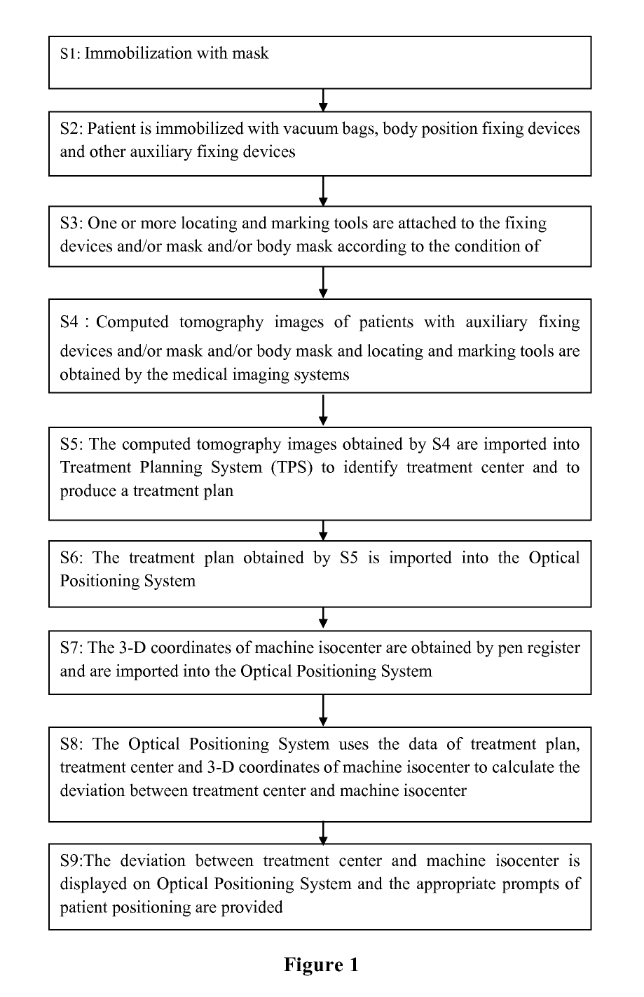

FIG. 2 is a flow chart comparing the procedures of the conventional method and the present invention;

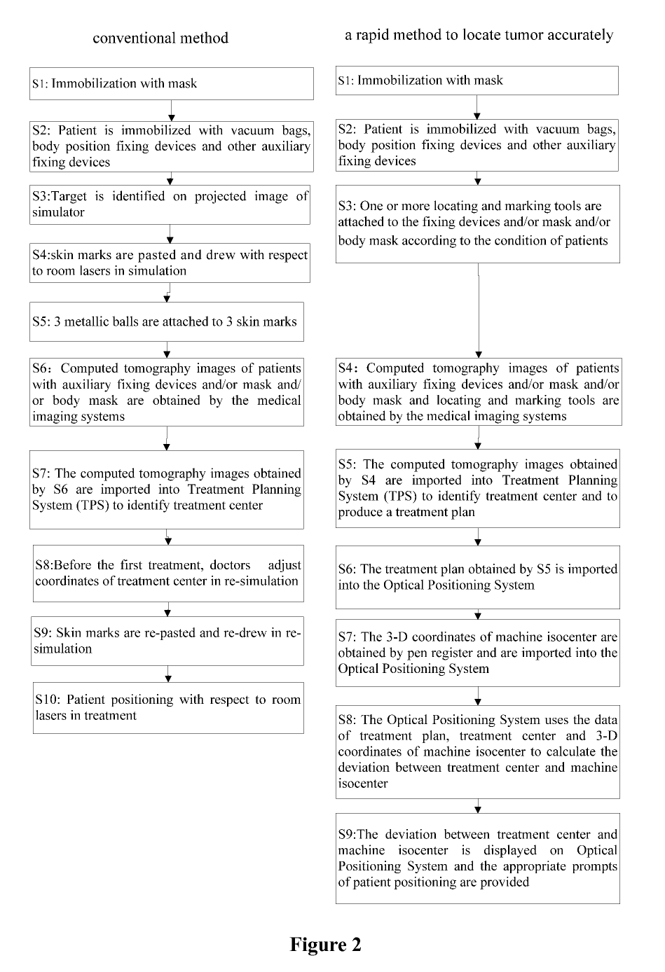

FIG. 3 is a flow chart comparing the positioning errors between the conventional method and the present invention.

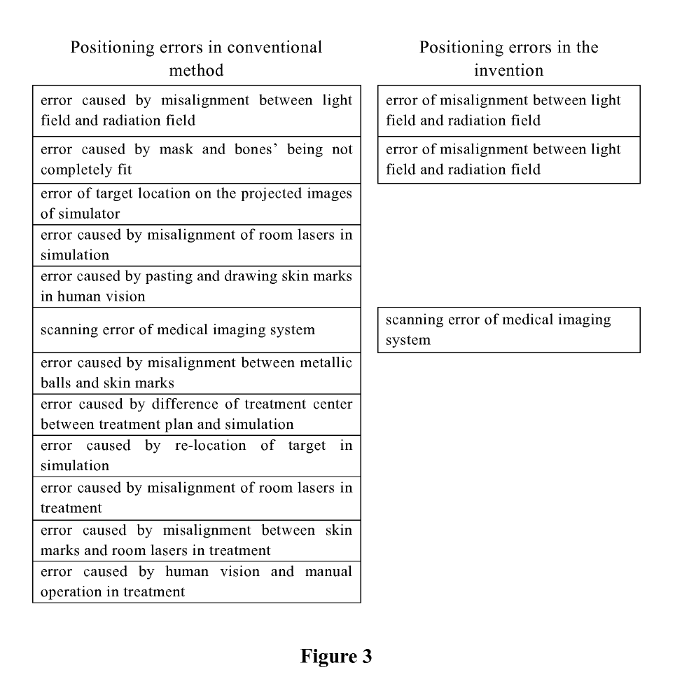

### DETAILED DESCRIPTION

In the following detailed description, reference is made to the accompanying drawings that form a part hereof, and in which is shown by way of illustration specific implementations that may be practiced. These implementations are described in sufficient detail to enable those skilled in the art to practice the implementations, and it is to be understood that other implementations may be utilized and that logical, mechanical, electrical and other changes may be made without departing from the scope of the implementation. The following detailed description is, therefore, not to be taken in a limiting sense.

The method to accurately and rapidly locate tumors can comprise an immobilization module, which ensures the consistency of patient positioning, verification, and the position of each treatment; a locating and marking module, wherein, the locating and marking tools can be mounted on auxiliary fixing devices for radiotherapy; a data input module, which obtains the data required for the calculation of Optical Positioning System in radiotherapy; and a positioning module, which calculates the deviation between the planned isocenter and the machine isocenter, and provides prompts of patient positioning.

The invention discloses a rapid method to locate tumors accurately, which comprises one or more of the following steps:
S1: Immobilization with mask
Patients with brain or head and/or neck tumor are immobilized with masks and patients with body tumor are immobilized with body masks.
S2: Patient is immobilized with vacuum bags, body position fixing devices and other auxiliary fixing devices.
S3: One or more locating and marking tools are attached to the fixing devices and/or mask and/or body mask according to the condition of patients.

The locating and marking tools are attached to at least one of the following locations: 1) auxiliary fixing devices; 2) body mask; 3) face mask.

In at least one embodiment of the invention, the locating and marking tool mentioned is the positioning ball, however, the technicians in this field could know that the marking objects in other shapes or other materials with an obvious infrared signature can take the place of the positioning ball without creative work. Therefore, similar technical solutions do not lie outside the range of the invention disclosure and claims.

The number of the locating and marking tools is preferably 6 or more to ensure that the translational or rotational changes of the locating and marking tools which when detected can fully indicate the changes of 6 degrees of freedom in three-dimensional space. If less than 6 locating and marking tools are used, the accuracy of the positioning can be insufficient. However, if more than 6 tools are used, the computational complexity will increase significantly although a higher accuracy can be obtained, making it impractical.
S4: Computed tomography images of patients with auxiliary fixing devices and/or mask and/or body mask and locating and marking tools are obtained by the medical imaging systems;
The positions of the locating and marking tools, human body and auxiliary fixing devices are obtained by a medical imaging system.
S5: The computed tomography images obtained by S4 are imported into Treatment Planning System (TPS) to identify the planned isocenter and to produce a treatment plan;
S6: The treatment plan obtained by S5 is imported into the Optical Positioning System;
S7: The 3-D coordinates of machine isocenter are obtained by a pen register, and are imported into the Optical Positioning System.
S8: The Optical Positioning System uses the data of the treatment plan, the planned isocenter and the 3-D coordinates of the machine isocenter to calculate the deviation between the planned isocenter and the machine isocenter.
The Optical Positioning System mentioned in the invention can use the data of the treatment plan, the planned isocenter and the 3-D coordinates of the machine isocenter to calculate the deviation between the planned isocenter and the machine isocenter in real time or non-real time.
S9: The deviation between the planned isocenter and the machine isocenter is displayed on Optical Positioning System, and the appropriate prompts of patient positioning are provided.

The prompts of patient positioning disclosed in the invention can comprise the manual operating instructions to instruct users how to manually adjust the parameters of the treatment couch, the gantry, etc; or/and the automatic operating instructions to give an automatic positioning control device instructions to automatically adjust the parameters of the treatment couch, the gantry, etc.

Compared to the conventional method, the rapid method to locate tumors accurately can simplify the procedure of the conventional method of patient positioning, and can greatly reduce the positioning errors caused by manual operations and repeated patient positioning. Consequently, it greatly improves the efficiency of tumor location and lowers the work strength of physicians and radiotherapists. Furthermore, the invention can provide real-time and non-real-time positioning correction instructions to users or an automatic positioning device to locate target properly, which improves the positioning accuracy of the entire course of patient positioning.

Note that, for the persons skilled in the art, the equivalent changes of the invention made under the guidance of the invention should be included in the scope of the invention claims.

What is claimed is:

1. A method to accurately and rapidly locate tumor, comprising the steps of:
using a immobilization module to ensure the consistency of patient positioning, verification and the position of each treatment, comprising one of the following: 1) auxiliary fixing devices;
2) body mask; 3) face mask;
using a locating and marking module comprising a locating and marking tool for locating and marking;
using a data input module to obtain the data required for a calculation of Optical Positioning System in radiotherapy; and
using a positioning module to calculate the deviation between the planned isocenter and the machine isocenter, and to provide prompts of patient positioning.

2. A method to accurately and rapidly locate tumor as described in claim 1, wherein, the locating and marking tools are attached to at least one of the following locations: 1) auxiliary fixing devices; 2) body mask; 3) face mask.

3. A method to accurately and rapidly locate tumor as described in claim 2, wherein the locating and marking tool is a positioning ball.

4. A method to accurately and rapidly locate tumor as described in claim 3, wherein the number of the locating and marking tools changes according to the condition of patients.

5. A method to accurately and rapidly locate tumor as described in claim 4, wherein, the number of the locating and marking tools is 6 and more.

6. A method to accurately and rapidly locate tumor as described in claim 1, wherein, the step of using data input module to obtain the data required for a calculation of Optical Positioning System in radiotherapy further comprise the following steps: 1) using a medical imaging system to obtain the positions of the locating and marking tools, human body and auxiliary fixing devices; 2) using the Treatment Planning System (TPS) to produce a treatment plan; 3) using the 3-D coordinates of the machine isocenter are obtained by a pen register

7. A method to accurately and rapidly locate tumor as described in claim 6, wherein the medical imaging system is a high-precision computed tomography scanner or a CT scanner.

8. A method to accurately and rapidly locate tumor as described in claim 1, wherein the step of using a positioning module to calculate the deviation between the planned isocenter and the machine isocenter, and to provide patient positioning further comprise the step of using Optical Positioning System to calculate the deviation between the planned isocenter and the machine isocenter, based on the information of the treatment plan, the planned isocenter and the 3-D coordinates of the machine isocenter; and the Optical Positioning System displays the deviation to users.

9. A method to accurately and rapidly locate tumor as described in claim 1, wherein the step of using positioning module further comprise the step of providing appropriate prompts for adjustment of patient positioning.

10. A method accurately and rapidly locate tumor as described by claim 9, wherein, the prompts of patient positioning further comprising the manual operating instructions to instruct users how to manually adjust the parameters of the treatment table, the gantry, etc; or/and the automatic operating instructions to give an automatic positioning control device instructions to automatically adjust the parameters of the treatment couch, the gantry, etc.

11. A method to accurately and rapidly locate tumor as described in claim 8, wherein, the Optical Positioning System can use the data of the treatment plan, the planned isocenter and the 3-D coordinates of the machine isocenter to calculate the deviation between the planned isocenter and the machine isocenter in real time or non-real time.

12. A method to accurately and rapidly locate tumor as described in claim 1, wherein, the immobilization module further comprise the step of immobilizing patients with vacuum bags, body position fixing devices and other auxiliary fixing devices.

13. A method to accurately and rapidly locate tumor as described in claim 1, wherein, the immobilization module further comprise the step of immobilizing patient with mask.

14. A system to accurately and rapidly locate tumor, comprising:
a immobilization module to ensure the consistency of patient positioning, verification and the position of each treatment;
a locating and marking module comprising a locating and marking tool for locating and marking;
a data input module to obtain the data required for a calculation of Optical Positioning System in radiotherapy; and
a positioning module to calculate the deviation between the planned isocenter and the machine isocenter, and to provide prompts of patient positioning.

15. A system to accurately and rapidly locate tumor as described in claim 14, wherein, the locating and marking tools are attached to at least one of the following locations: 1) auxiliary fixing devices; 2) body mask; 3) face mask.

16. A system to accurately and rapidly locate tumor as described in claim 15, wherein the locating and marking tool is a positioning ball.

17. A system to accurately and rapidly locate tumor as described in claim 16, wherein the number of the locating and marking tools changes according to the condition of patients.
